(19) United States
(12) Patent Application Publication
(10) Pub. No.: US 2023/0399456 A1
(43) Pub. Date: Dec. 14, 2023

(54) CARBON-IODINE CONJUGATED POLYMER AND PREPARATION THEREOF, AND USE THEREOF FOR PREPARING LOCALIZATION MARKER

(71) Applicant: Huazhong University of Science and Technology, Wuhan, Hubei (CN)

(72) Inventors: Liang LUO, Wuhan, Hubei (CN); Mingming YIN, Wuhan, Hubei (CN); Xiaoming LIU, Wuhan, Hubei (CN); Fanling MENG, Wuhan, Hubei (CN)

(21) Appl. No.: 18/256,082

(22) PCT Filed: Mar. 8, 2022
(86) PCT No.: PCT/CN2022/079639

§ 371 Date: Jun. 6, 2023

(30) Foreign Application Priority Data
Nov. 30, 2021 (CN) 202111441503.9

(51) Int. Cl. C08G 61/04; A61K 49/04

(52) U.S. Cl. CPC C08G 61/04; A61K 49/0442; C08G 2261/11; C08G 2261/146; C08G 2261/18; C08G 2261/3327; C08G 2261/3328; C08G 2261/1432

(57) ABSTRACT

Described are a carbon-iodine conjugated polymer and preparation thereof, an imaging marker thereof, and uses thereof for preparing a localization marker, and belongs to the technical field of imaging markers. The conjugated structure enables the polymer to have strong absorption in a visible light region, and high iodine content of up to 84.1% corresponds to the strong imaging ability thereof. During surgery, on the basis of the dual guidance of a polymer-based image marker and naked eye observation, the marker can better facilitate determination of tumor resection margins, achieving precise resection of tumors and minimizing damage to surrounding normal tissues. During cyberknife-based treatment, the polymer can replace clinical gold markers to provide ray marker guidance. Absence of metal artifacts improves ray imaging quality and the accuracy of radiation dose distribution, good biocompatibility enhances the stability of the relative position of the marker, and radiotherapy side effects can be further reduced.

### TECHNICAL FIELD

The present invention belongs to the technical field of imaging markers, and more specifically, relates to a carbon-iodine conjugated polymer and preparation thereof, and a use thereof for preparing a localization marker.

### BACKGROUND ART

Cancer is the major cause of morbidity and mortality worldwide. There are approximately 14 million new cancer patients and eight million people dying from cancer-related diseases every year. In consideration of the high risk and high mortality rate of cancer, researchers in the world are making an ongoing effort to develop more precise and rapid diagnosis and treatment methods to fight cancer. In clinical cancer treatment, most cancer treatment methods (such as chemotherapy, radiotherapy and surgery) are partially successful but have certain limitations. Moreover, these cancer treatment methods tend to damage surrounding healthy tissues, ultimately affecting the survival time of patients. Hence, precise treatment of tumors is considered an important direction for future development. Although there are many reports of targeted drugs in chemotherapy, the issues of systemic toxicity and multi-drug resistance in patients are still unavoidable problems. Therefore, people place their hopes on precise localization of tumors during radiotherapy and precise resection of tumors during surgery. The key to achieving such goals lies in precise marking and real-time tracking of tumor contours under the precise guidance of medical imaging.

CT images of tumors are one of the most important references in the processes of tumor diagnosis and treatment planning. CT image analysis can precisely show the three-dimensional shape and relative position of a tumor in the body of a patient. However, during actual surgery or radiotherapy, tumor position determination requirements are extremely high, and it is difficult to maintain the relative stillness between the tumor and the patient's appearance due to normal respiratory movement of the human body or the impact of surgical procedures. To achieve precise tumor localization during surgery or radiotherapy, it is necessary to additionally introduce CT markers for real-time tracking of the status and relative position of the tumor as well as surrounding tissues and organs, thereby ultimately achieving precise tumor treatment. Based on clinical needs, the requirements for CT markers mainly focus on high CT imaging quality, easy identification with the naked eye, stable nature and stable relative position during treatment, good biocompatibility, and applicability to different tumor treatment scenarios.

### SUMMARY OF THE INVENTION

The present invention solves the technical problems of poor imaging quality, unstable relative position, and poor biocompatibility of imaging markers in the prior art. Provided in the present invention are a conjugated polymer containing a polydiacetylene (PDA) backbone but with only iodine atom substituents, a synthesis method thereof, and a use thereof for precise in-vivo marker-guided surgical resection and radiotherapy. The marker provided by the present invention has high iodine content and ultra-high X-ray absorption efficiency, contains nanofibers which tend to self-assemble to form clusters so as to easily maintain stability and prevent diffusion at specific sites, and provides good biocompatibility.

According to a first aspect of the present invention, a carbon-iodine conjugated polymer is provided. The structural formula of the carbon-iodine conjugated polymer comprises the structure shown in formula II, formula II being:

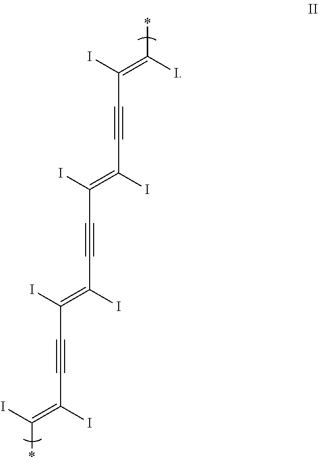

Preferably, the structural formula of the carbon-iodine conjugated polymer is as shown in formula I or formula II:

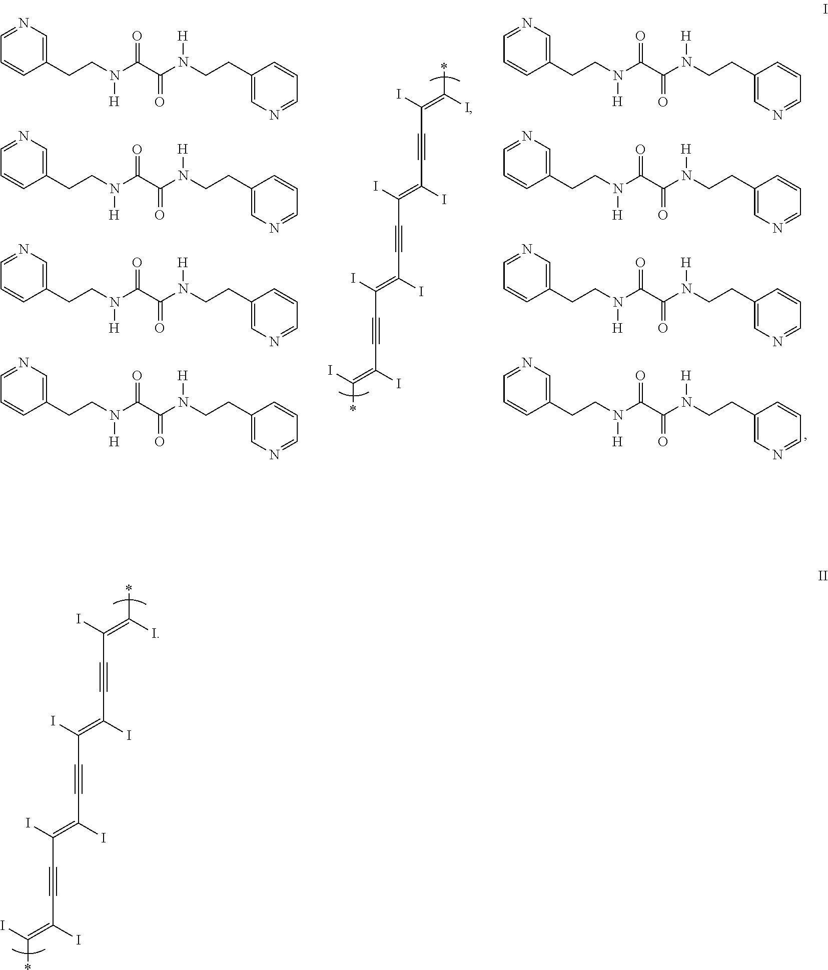

According to another aspect of the present invention, a synthesis method of a carbon-iodine conjugated polymer having the structure shown in formula I is provided. A reaction formula is as follows:

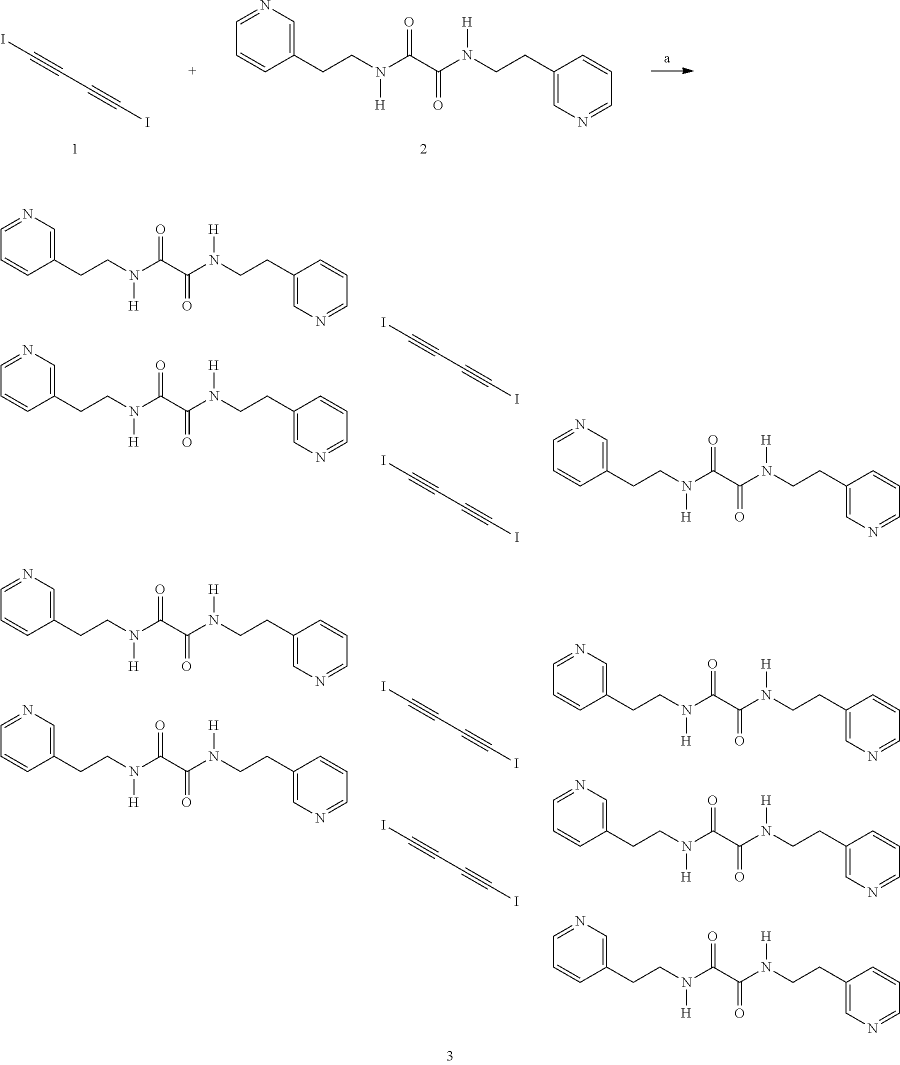

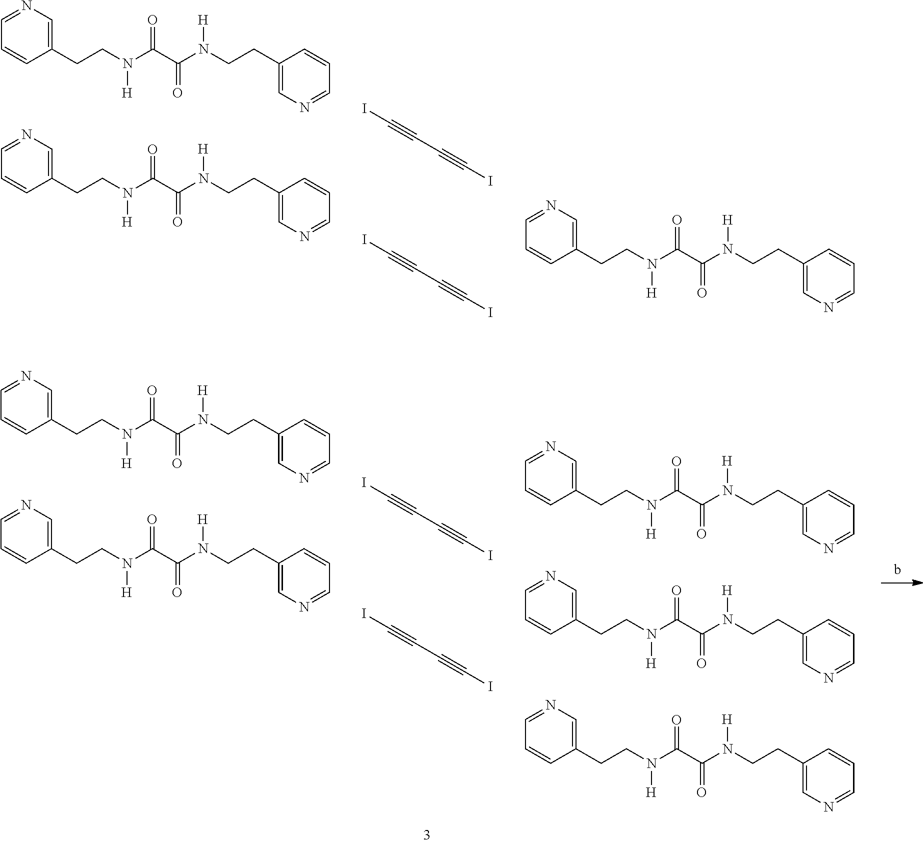

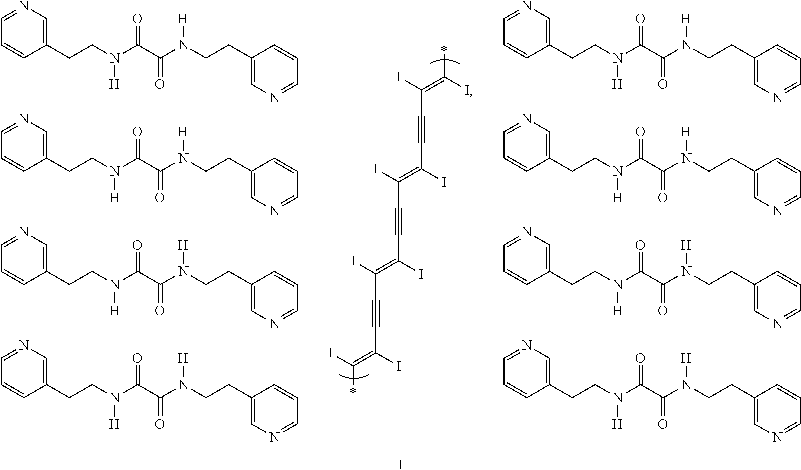

the synthesis method comprises the following steps:
a, adding iodine substituted hexadine shown in formula 1 and a ligand shown in formula 2 into methanol, ethanol, or isopropanol, the ligand and the iodine substituted hexadine being arranged regularly to form an intermediate shown in formula 3; and
b, carrying out topochemical polymerization on iodine atoms on the iodine substituted hexadine in formula 3 and pyridine group nitrogen atoms at a terminal of the ligand in formula 2 through halogen bonding to obtain a carbon-iodine conjugated polymer having the structure shown in formula I;
preferably, in step a, after being added to methanol, ethanol, or isopropanol, the iodine substituted hexadine shown in formula 1 and the ligand shown in formula 2 are kept at −30° C. to −10° C. for 5 to 10 days, and then kept at 10° C. to 30° C. for 5 to 12 hours.

According to another aspect of the present invention, a synthesis method of a carbon-iodine conjugated polymer having the structure shown in formula II is provided. A reaction formula is as follows:

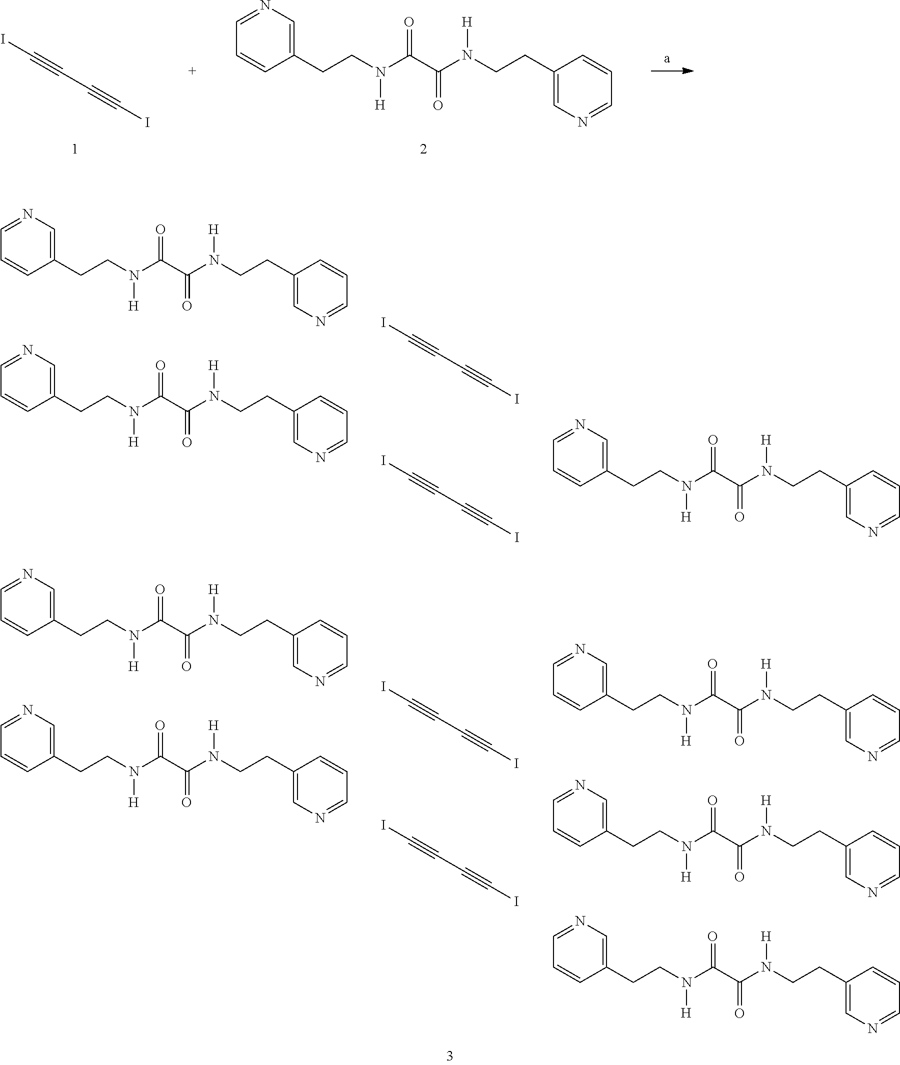

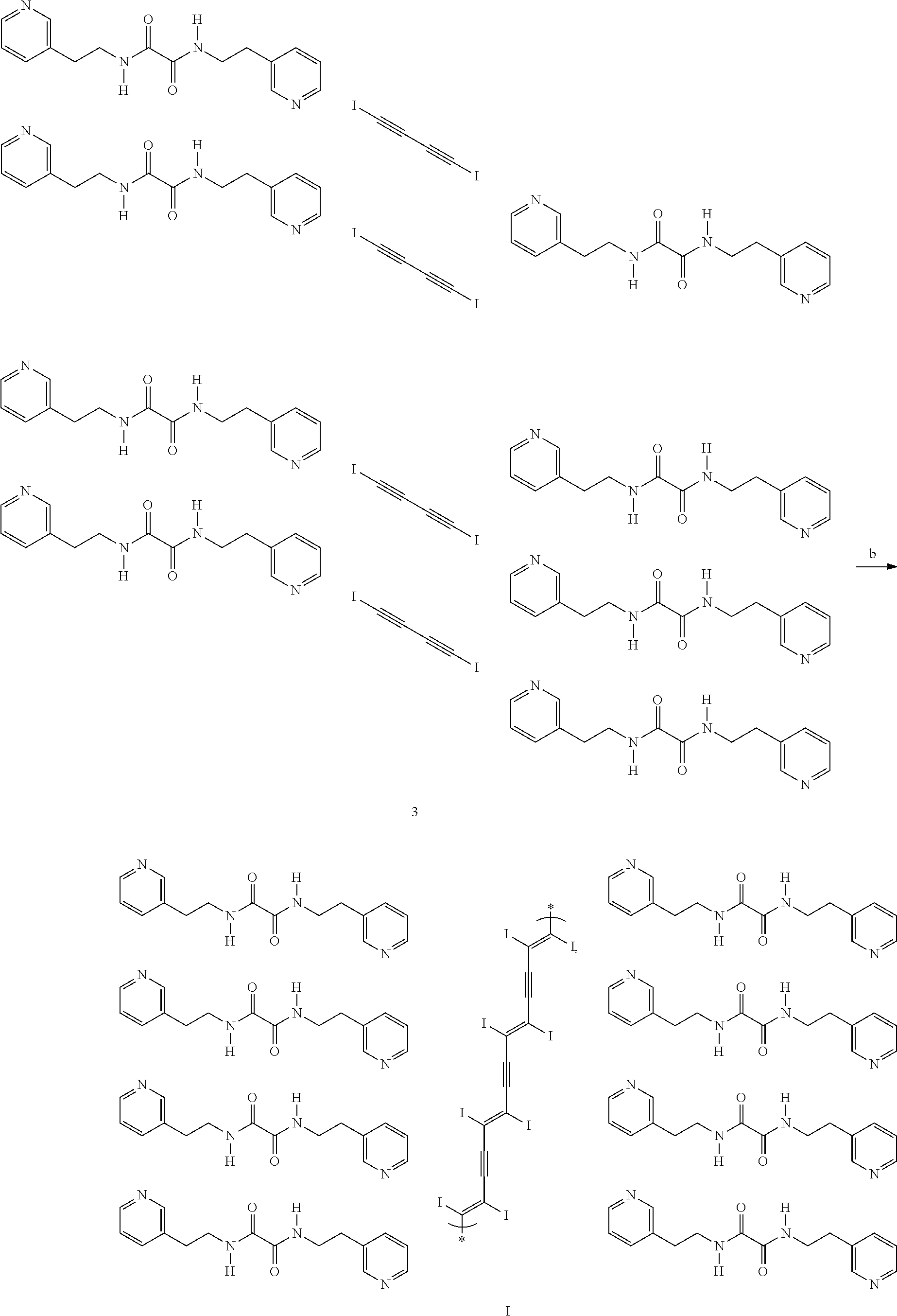

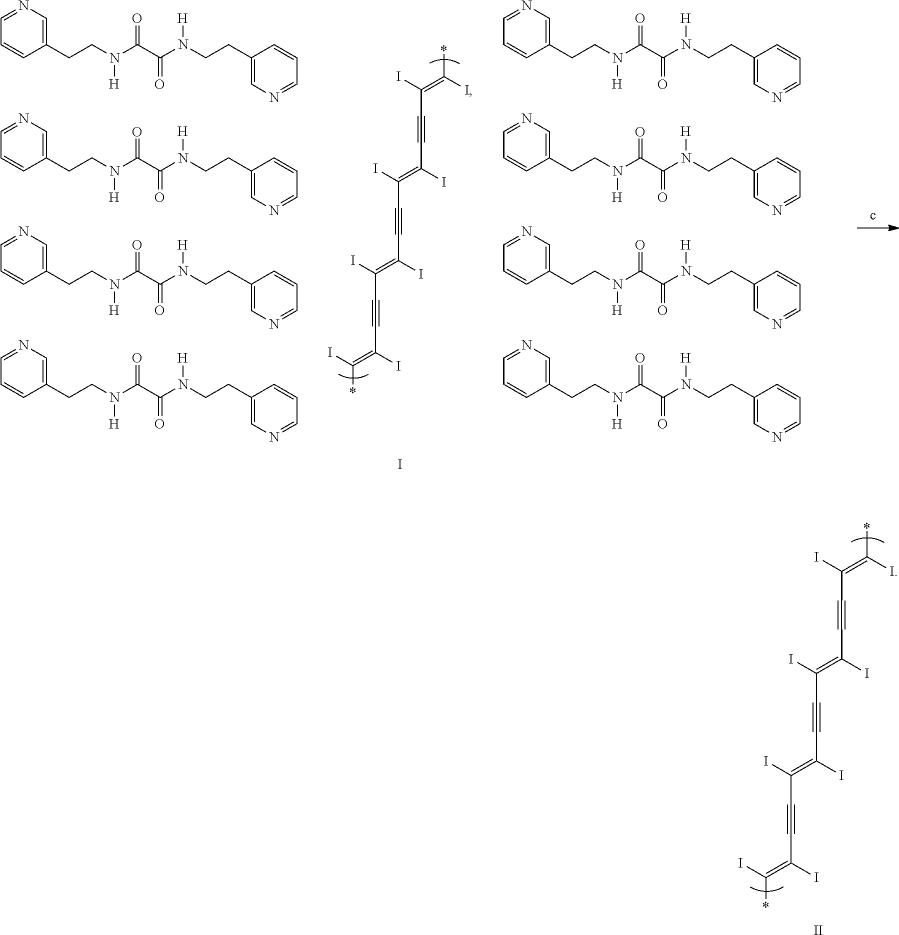

the synthesis method comprises the following steps:
a, adding iodine substituted hexadine shown in formula 1 and a ligand shown in formula 2 into methanol, ethanol, and isopropanol, the ligand and the iodine substituted hexadine being arranged regularly to form an intermediate shown in formula 3;
b, carrying out topochemical polymerization on iodine atoms on the iodine substituted hexadine in formula 3 and pyridine group nitrogen atoms at a terminal of the ligand in formula 2 through halogen bonding to obtain a conjugated carbon iodine polymer having the structure shown in formula I; and
c, adding the carbon-iodine conjugated polymer having the structure shown in formula I and obtained in step b into methanol or a dilute hydrochloric acid solution and washing the same, performing a centrifugal operation to remove a supernatant, and carrying out vacuum drying to obtain a carbon-iodine conjugated polymer having the structure shown in formula II.

According to another aspect of the present invention, a method for preparing an aqueous dispersion containing a carbon-iodine conjugated polymer having the structure shown in formula II is provided, and comprises: causing a carbon-iodine conjugated polymer having the structure shown in formula II and an amphiphilic polymer to undergo ultrasonic stripping in water, a hydrophobic end of the amphiphilic polymer being an alkyl chain having a carbon atomic number greater than or equal to 10, and a hydrophilic end thereof being polyethylene glycol; and causing the carbon-iodine conjugated polymer having the structure shown in formula II to be linked to the amphiphilic polymer through an intermolecular force to form an aqueous dispersion containing the carbon-iodine conjugated polymer having the structure shown in formula II, the carbon-iodine conjugated polymer having the structure shown in formula II being as follows:

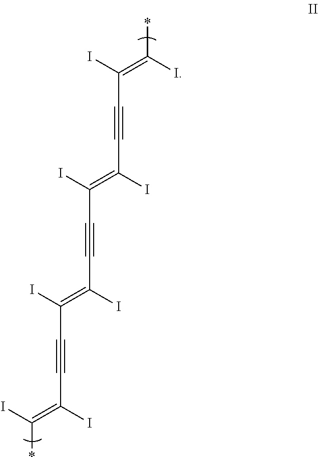

According to another aspect of the present invention, provided is an aqueous dispersion containing a carbon-iodine conjugated polymer having the structure shown in formula II prepared by the method.

According to another aspect of the present invention, provided is a use of the carbon-iodine conjugated polymer or the aqueous dispersion containing a carbon-iodine conjugated polymer having the structure shown in formula II, the use being specifically for preparing a localization marker.

Preferably, the localization marker is an imaging marker.

Preferably, the imaging marker is an X-ray marker.

Preferably, the X-ray marker is a CT imaging marker.

According to another aspect of the present invention, provided is a use of the carbon-iodine conjugated polymer, the use being specifically for preparing a body surface auxiliary marker patch.

In general, compared with the prior art, the above technical solutions conceived by the present invention have the following technical advantages:
(1) Precise treatment of tumors relies on guidance of CT images, and suitable CT markers are often used to facilitate continuous tracking during precise tumor treatment. Disclosed in the present invention is a novel CT marker employing a carbon-iodine conjugated polymer PIDA. The conjugated structure enables PIDA to have strong absorption in a visible light region, and high iodine content of PIDA of up to 84.1% corresponds to the super-strong CT imaging ability thereof. During tumor surgery, based on the dual guidance of a PIDA-based CT image marker and naked eye observation, a PIDA marker can better facilitate determination of tumor resection margins, thereby achieving precise resection of tumors and minimizing damage to surrounding normal tissues. During cyberknife-based tumor treatment, PIDA can replace the clinical gold marker to provide CT marker guidance. The absence of metal artifacts improves CT imaging quality and improves the accuracy of radiation dose distribution, good biocompatibility enhances the stability of the relative position of the PIDA marker, and radiotherapy side effects can be further reduced. In addition, unlike the clinical gold marker which permanently stays in the human body after use, PIDA performs functions during treatment and gradually bio-degrades in a few months after treatment. Compared with corresponding clinical markers, PIDA can show corresponding functions in various tumor treatment modes, and can make up for the defects and shortcomings of existing markers, thereby better meeting the needs of clinical use.
(2) Disclosed in the present invention is a use of a conjugated polymer containing a polydiacetylene (PDA) backbone but with only iodine atom substituents, for precise in-vivo marker-guided surgical resection and radiotherapy. The conjugated polymer in the present invention has high absorption efficiency for visible light and X-rays, good biocompatibility, excellent marking stability, biodegradability, and a wide range of applications. The use of these PIDA properties achieves a revolutionary breakthrough in precise surgery and radiotherapy of tumors, thereby greatly promoting the development of precise tumor treatment.
(3) The conjugated polymer in the present invention itself is a nanofiber structure having iodine content of up to 84.1%. The ultra-high iodine content endows the conjugated polymer with ultra-high X-ray absorption efficiency, and nanofibers tend to self-assemble to form clusters so as to maintain stability and prevent diffusion at specific sites. Iodine atoms are directly linked to a highly-conjugated carbon chain, such that the conjugated polymer is provided with a strong molar extinction coefficient, making the conjugated polymer dark blue or even black, and facilitating naked eye observation.
(4) In the present invention, by introducing an amphiphilic polymer (such as C18-PMH-PEG), a PIDA block is regulated to form a PIDA nanofiber dispersion, so as to achieve local injection to a specific site of the body through an endoscope, thereby greatly expanding the range of applications of markers.
(5) The iodine atom substituents of the conjugated polymer in the present invention are linked to both sides of a carbon-carbon double bond of a conjugated backbone. Such conjugated structure allows for low bond energy of a covalent bond between carbon and iodine, and breakage and deiodination tend to occur under the stimulation of Lewis bases and the like.
(6) Disclosed in the present invention is a use of a conjugated polymer containing a polydiacetylene (PDA) backbone but with only iodine atom substituents, for precise in-vivo marker-guided surgical resection and radiotherapy. As a pure carbon-iodine non-metallic organic polymer, PIDA has simple structural constitution, does not contain any other heavy atoms, and has good biocompatibility. The conjugated structure of the present invention facilitates gradual degradation of PIDA under external stimulation.

### BRIEF DESCRIPTION OF THE DRAWINGS

FIG. 1 shows preparation and characterization of PIDA.

FIG. 2 shows representations of the stability of PIDA under ionizing radiation.

FIG. 3 shows imaging effects of PIDA in in-vitro tissues.

FIG. 4 shows imaging effects of PIDA in rat muscles.

FIG. 5 shows a resection surgery guided by PIDA for marking tumor contours of a rat.

FIG. 6 shows a resection surgery guided by PIDA for marking the tumor interior of a rat.

FIG. 7 shows simulated imaging effects inside the human body when PIDA is applied to a human model.

FIG. 8 shows internal organ distribution of a human model.

FIG. 9 shows representations of the biological safety of PIDA in rat liver.

FIG. 10 shows the implementation process in which PIDA is applied to cyberknife tracking.

FIG. 11 is a real-life image of implantation of PIDA into a rat under CT image guidance.

FIG. 12 is a real-life image of implantation of PIDA into a rat for cyberknife tracking.

FIG. 13 is a real-life image of implantation of PIDA into a beagle for cyberknife tracking.

FIG. 14 shows representations of in-vivo degradation of PIDA and effects thereof on individual organs.

FIG. 15 shows blood analysis after implantation of PIDA into rats.

FIG. 16 is a schematic diagram of PIDA-guided surgical treatment and cyberknife radiotherapy.

### DETAILED DESCRIPTION

To make the purpose, technical solution, and advantages of the present invention clearer, the present invention is further described in detail below in connection with the accompanying drawings and examples. It should be appreciated that the specific examples described here are used merely to explain the present invention and are not used to define the present invention. In addition, the technical features involved in various embodiments of the present invention described below can be combined with each other as long as a conflict is not constituted therebetween.

### Example 1: Polymer Design and CT Imaging Performance Characterization

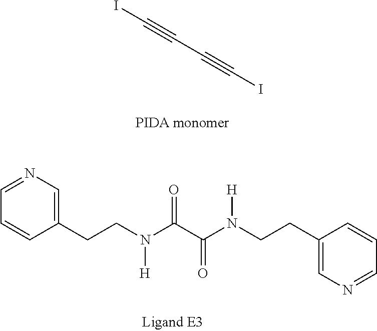 [SMILES: IC#CC#CI ; O=C(NCCc1cccnc1)C(=O)NCCc1cccnc1]

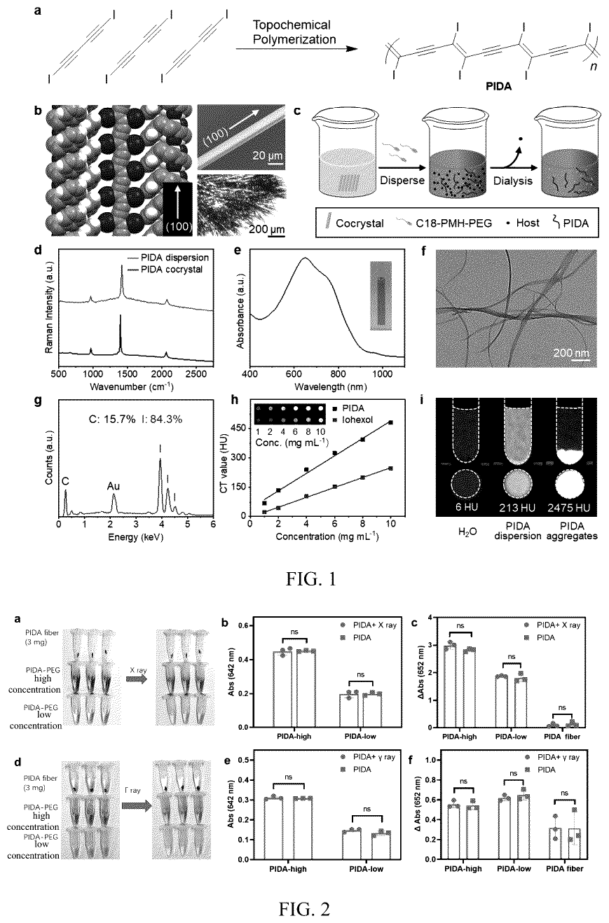

According to a ligand-receptor cocrystal polymerization synthesis method, in the present invention, single crystals of a PIDA monomer and a ligand E3 were cultured in methanol, so that topochemical polymerization could be achieved at room temperature to obtain a PIDA-E3 cocrystal (a in FIG. 1). A three-dimensional structure between the conjugated polymer PIDA and the small molecule ligand E3 was confirmed by single crystal X-ray diffraction analysis (b in FIG. 1). The distance between iodine atoms on PIDA side chains and pyridine group nitrogen atoms at a terminal of the ligand E3 was 2.925 Å, and the two interacted through strong halogen bonding. The small molecule E3 itself was arranged orderly through hydrogen bonds between oxalic acid amide structures, and the distance was the same as that of repeating units on PIDA, i.e., 4.957 Å. A PIDA backbone had a conjugated structure with alternating carbon-carbon double bonds and carbon-carbon triple bonds. A side chain iodine atom substituent was linked to the end of the carbon-carbon double bond, and C—C—I had an included angle of 113 degrees. The combination between the PIDA monomer and the ligand E3 was conducive to 1,4 polymerization of the carbon-carbon triple bonds, resulting in an initial PIDA monomer-ligand E3 being light blue when a crystal was just formed at −20° C. Rapidly polymerization could be achieved within a few hours at room temperature. The final polymer had a metallic luster due to the high degree of polymerization and a completely planar backbone conformation.

In order to adjust the cocrystal morphology and physical properties of PIDA for subsequent use, an amphiphilic polymer C18-PMH-PEG was introduced in the present invention. A substituent at one end of the polymer was a hydrophobic long alkyl chain, while a substituent at the other end of the polymer was a hydrophilic PEG long chain, so that a good dispersion effect was achieved on carbon nanotubes. A blue dispersion could be obtained by performing ultrasonic stripping on the PIDA cocrystal and C18-PEM-PEG in a mass ratio of 1:1 in pure water, and the small molecule ligand dissolved in water was removed by dialysis to obtain a blue aqueous dispersion of the PIDA polymer (c in FIG. 1).

Due to the large polarizability and highly-conjugated plane of a polydiacetylene backbone, the PIDA cocrystal had a very strong Raman scattering intensity, and three main Raman characteristic peaks 967 cm⁻¹, 1396 cm⁻¹, and 2064 cm⁻¹ corresponded to stretching vibrations of C—C, C═C, and C≡C respectively. The locations of three main Raman characteristic peaks of the PIDA dispersion were 966 cm⁻¹, 1417 cm⁻¹, and 2075 cm⁻¹, which, in comparison, were closer to PIDA fiber than the PIDA cocrystal (d in FIG. 1). This indicated that the small molecule ligand was removed, and the main component of the dispersion was PIDA that maintained alternate conjugation of the carbon-carbon double bonds and the carbon-carbon triple bonds.

The blue PIDA aqueous dispersion corresponded to an ultraviolet-visible absorbance peak at 652 nm (e in FIG. 1). TEM showed that the microstructure thereof is a dispersed nanofiber structure with a diameter of about 1-3 um and a diameter of about 30 nm. The particle size thereof determined by DLS was on the order of hundreds of nanometers, and the dispersion gradually aggregated into fibrous clusters as the concentration increased (f in FIG. 1). The particle size gradually increased, and the surface charge was gradually closer to electrical neutrality, which was related to the fact that the nanofibers tend to aggregate into clusters at the microscopic level. The elemental composition of PIDA characterized by energy dispersive X-ray spectroscopy (EDX) analysis was C: 15.7%, I: 84.3% (g in FIG. 1), which was consistent with the theoretical value of PIDA.

An X-ray attenuation number is positively correlated with the atomic number and the density. Heavy atom iodine with an atomic number of 53 has a very strong X-ray attenuation ability. Currently, clinically used CT contrast agents are all small molecules using triiodobenzene as a core. Improving effective iodine loading is one of the important development directions of the CT contrast agents. However, the iodine content of current clinical CT contrast agents (for example, iohexol being 46.4%, iopromide being 48.1%, and iodixanol being 49.1%) does not exceed 50%. The iodine content (84.1%) of PIDA makes it an excellent CT contrast agent. By comparing PIDA of different concentrations with iohexol, the most commonly used medical CT contrast agent at present, it was found that the CT contrast ability gradually increased with the increase of the sample concentration. Even at very low concentrations, CT intensity maintained a good linear relationship with the PIDA concentration. In addition, since the X-ray attenuation ability of iodine is independent of the molecular structure environment thereof, the iodine content of PIDA was 1.81 times that of iohexol, and the measured imaging efficiency was 1.76 times that of iohexol (h in FIG. 1). Furthermore, when PIDA nanofibers were locally condensed into fibrous clusters, the local iodine density thereof increased greatly, and the CT signal intensity directly increased by an order of magnitude from 213 HU to 2475 HU (i in FIG. 1).

PIDA was applied to CT imaging of in-vitro tissues to explore the use of PIDA aggregation-induced CT enhancement in the in-vitro tissues. The PIDA dispersion and iohexol with the same iodine content were subcutaneously injected into pigs. In a contrast test of CT enhancement detection, it was found that with the same iodine content, after injection of iohexol into a fat layer and a muscle layer of pork, there were significant diffusing phenomena, resulting in weak CT imaging signals. After injection of PIDA, bright spots were formed locally, and CT signals were strong. The CT signal intensity of PIDA in the muscle layer (87.8 HU) was 4 times that of the corresponding iohexol CT signal intensity (21.7 HU). While in a relatively denser muscle layer, the CT signal intensity of PIDA in the muscle layer (251.7 HU) was 17 times that of the corresponding iohexol CT signal intensity (14.5 HU) (FIG. 3). This was because the PIDA nanofibers did not diffuse all around like small-molecule iohexol in a physiological environment, but mainly spontaneously aggregated locally at an injection site, so as to achieve local CT enhancement under the condition of low iodine concentration. Therefore, the ultra-high iodine content of PIDA endowed it with super-strong CT imaging ability, and the aggregation and agglomeration properties of the PIDA nanofibers themselves could further improve this ability.

To verify the stability of PIDA under different radiation conditions, in the present invention, PIDA in different states was placed at an X-ray machine. The testing conditions of the X-ray machine were 90 kV, and 4 mAs. A total of 50 tests were carried out, and a cumulative radiation dose was 1198.1 uGym². Changes in the absorbance peak of PIDA in the PIDA dispersion were verified by UV-visible absorption, and the concentration of iodine ions that might be precipitated was verified by introducing TMB and H₂O₂ reagents. Both tests showed that PIDA remained stable during testing (a, b, and c in FIG. 2). Likewise, in order to verify the influence of gamma rays on PIDA in normal radiotherapy, samples were treated with a cumulative radiation dose of gamma rays of 2 Gym² in the present invention, and the result also showed the stability of PIDA (d, e, and fin FIG. 2). The result demonstrated the stability of PIDA as a fiducial marker in CT diagnosis and radiotherapy.

### Example 2: Multiple In-Vivo Local Marking of Animals

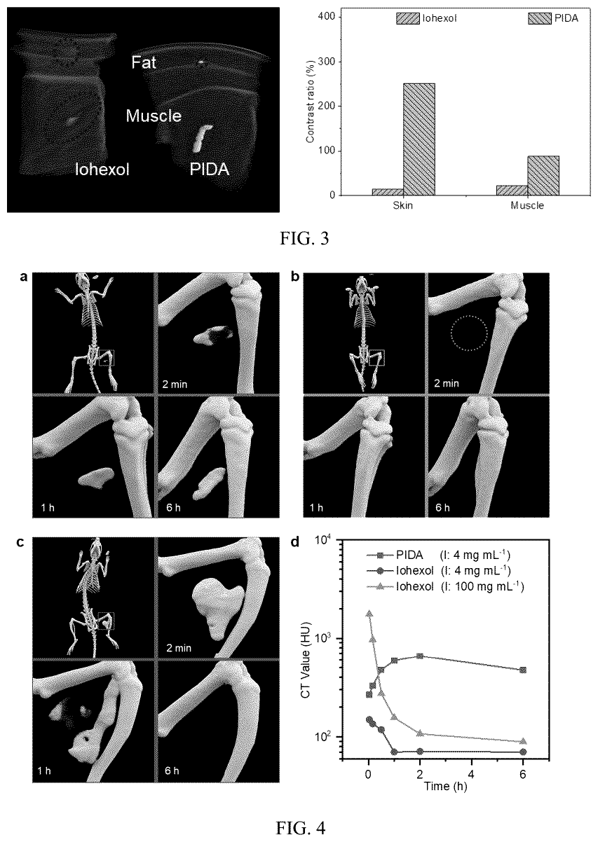

In order to verify whether the super-strong CT imaging effect of PIDA is practically applicable, the PIDA dispersion and iohexol with the same iodine content were injected locally into leg muscles of a rat respectively under CT guidance in the present invention. For effective CT marking, the CT signal intensity thereof should be 2 times or more that of background tissues. A background CT signal of rat muscle tissues is about 50 HU, so the CT marking signal intensity exceeding 100 HU can be regarded as effective CT marking. The result showed that the intramuscular injection site in the PIDA group showed a significant CT enhancement effect. Considering the overall effective time of clinical preoperative and intraoperative procedures, it was found that PIDA maintained a strong CT enhancement effect within 6 hours (a in FIG. 4). In the corresponding iohexol group, no local enhancement was observed at the intramuscular injection site during the entire process (b in FIG. 4).

To further verify the high efficiency and location stability of PIDA CT imaging, in the present invention, iohexol with the iodine content being increased by 25 times was injected into a corresponding leg muscle site of a rat. The result showed that the injection site and a large circle around the injection site initially showed a strong CT imaging effect, but such enhancement effect quickly diminished and gradually diffused towards the periphery of the injection site, and completely disappeared within 6 hours (c in FIG. 4). The curve of CT signal intensity over time showed that a 5 mg/ml PIDA dispersion could achieve long-term effective CT marking, while iohexol with the same iodine content basically had no marking effect, and when iohexol with ultra-high concentration was used, the signal intensity attenuated too quickly and the marking location was not fixed (d in FIG. 4). The overall experimental result showed that, compared with the medical CT contrast agent iohexol, PIDA had a high-efficiency CT marking effect and long-term stability at a target site during in-vivo local injection, and could better meet clinical marking requirements.

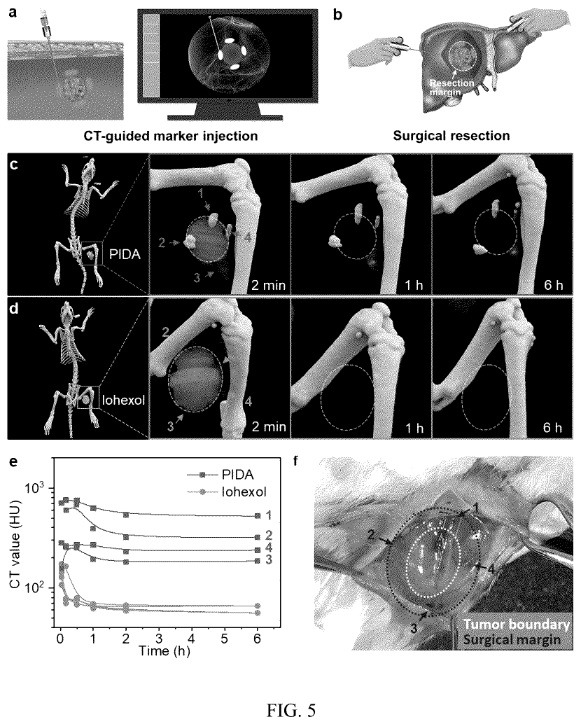

After confirming that PIDA has a good CT marking effect in both solutions and muscle tissues, the present invention explored the feasibility of using PIDA for tumor marking to guide surgical resection. In the present invention, PIDA was injected around a tumor in a rat under CT guidance. On the one hand, the CT imaging capability of PIDA was utilized to realize real-time CT guidance during surgical tumor resection (a in FIG. 5). On the other hand, color marking of PIDA itself was utilized to mark a tumor contour (b in FIG. 5). Tracking of CT signals at the injection site at different times showed that, compared with iohexol which rapidly disappeared and could not perform effective CT marking functions (d in FIG. 5), PIDA marking showed very high time stability in terms of the signal intensity and the relative position thereof around the tumor, and could accurately delineate the tumor contour (c and e in FIG. 5). After 24 hours, the rat was dissected, and PIDA markers distributed around the tumor could be easily found through epidermal mucosa (f in FIG. 5). Based on the dual guidance of tumor contour CT images and naked eye observation in surgical resection, PIDA could better facilitate determination of tumor resection margins, thereby achieving precise resection of tumors and minimizing damage to surrounding normal tissues.

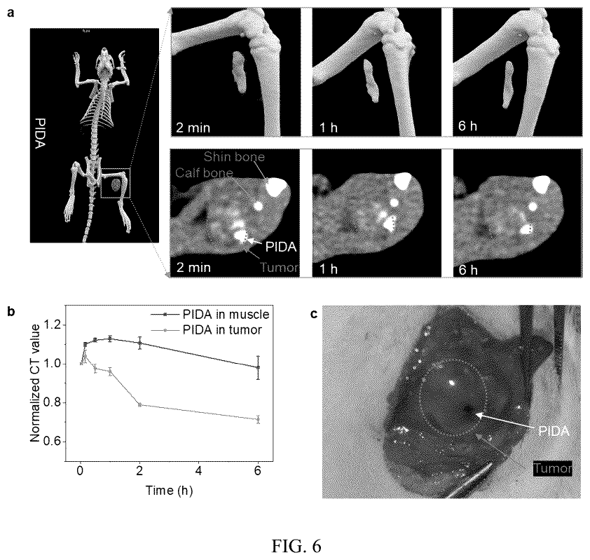

In addition to delineating the margins of large tumors, it is also an important problem to determine the relative position of small tumors such as lymph node metastatic tumors in the body during surgery. It is difficult to find the specific locations of small tumors confirmed by CT images during surgery. For such a usage scenario, in the present invention, the PIDA dispersion was directly injected into the tumor under CT guidance, so as to directly perform CT marking and naked-eye visible color marking on the tumor itself. CT imaging results at different times and the final anatomical observation from experiments also conformed to the expectations of the present invention. PIDA inside the tumor maintained effective CT marking (a in FIG. 6), and the dark PIDA location could be easily distinguished by the naked eye during tumor resection (c in FIG. 6). More interestingly, it was found in the present invention that the PIDA dispersion was only distributed and aggregated inside the tumor contour. Even if being injected at the margin of the tumor, the dispersion would not diffuse to any normal tissue site. At the same time, changes in the CT imaging effect of PIDA at different time points inside the tumor showed significant differences from that in normal tissues, and PIDA showed a stronger metabolic process inside the tumor (b in FIG. 6). The reason was the unique EPR effect of the tumor, i.e., the rich blood supply system, and imperfect lymphatic return led to the internal retention effect of the tumor. This provided more favorable conditions for PIDA to be used for local marking resection of the tumor.

### Example 3: PIDA Marking for Cyberknife Treatment

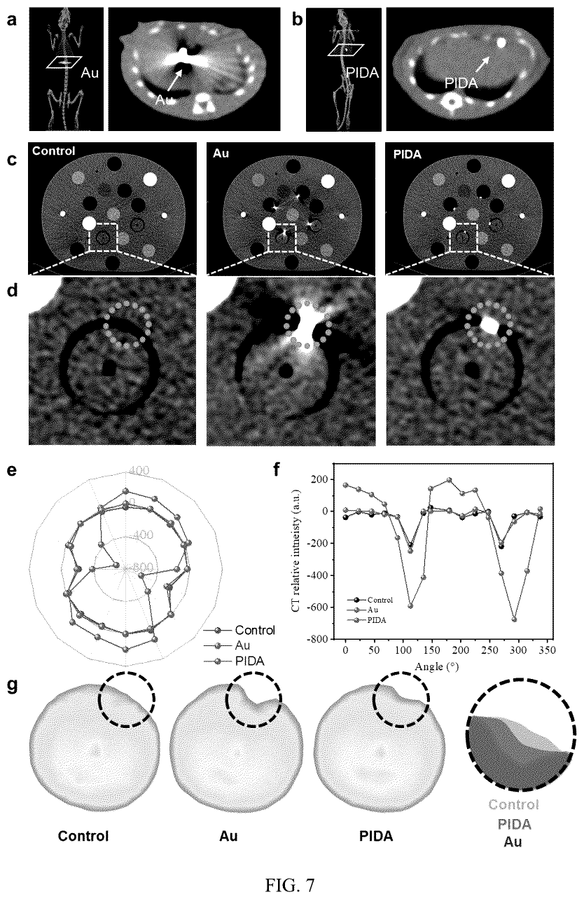

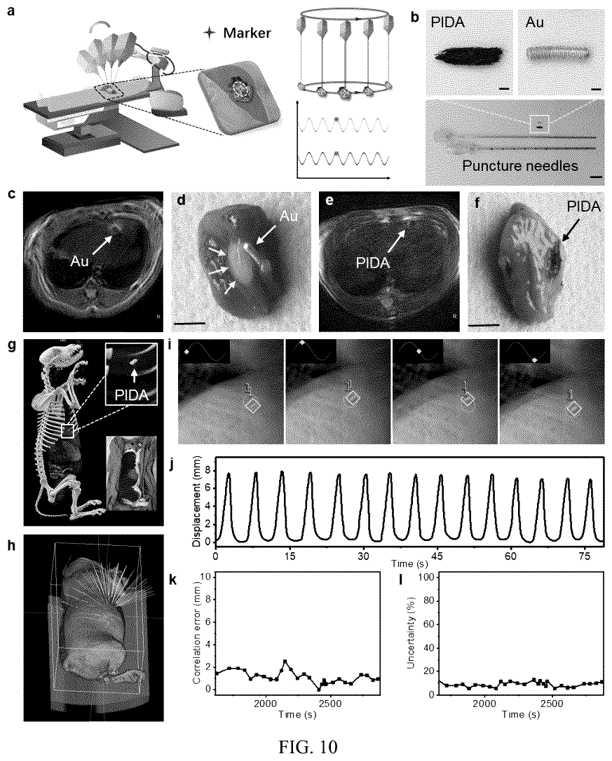

In addition to surgical resection of tumors, radiation is also an important means for tumor treatment. The most cutting-edge precise radiotherapy, that is, cyberknife treatment, relies on accurate CT localization of implanted markers (a in FIG. 10). At present, although gold elemental markers commonly used in clinical practice can meet the requirements of strong CT signals and relatively stable location of CT localization marking, local edema can be easily caused, which in turn leads to localization deviation. The biocompatibility is poor, and the markers retain permanently in the body and are difficult to degrade. CT metal artifacts are serious (a in FIG. 7), which affects the CT imaging quality and the planning of subsequent radiation dose distribution. Such problems reduce cyberknife treatment effects, and limit the further popularization and use of the cyberknife treatment. Based on this, in the present invention, the gold markers were replaced by PIDA solid fibers, and the PIDA solid fibers were implanted into the liver of a rat under CT guidance for corresponding CT marking for guiding subsequent cyberknife treatment (b in FIG. 10). The CT imaging result showed that while PIDA achieved efficient CT imaging marking, there was basically no interference from metal artifacts corresponding to gold (b in FIG. 7). An MRI image after 24 hours verified that local edema was induced by an implanted gold marker (c in FIG. 10), while PIDA caused no significant difference (e in FIG. 10). The dissection result (FIG. 9) and the analysis of blood inflammatory response in the table below further supported this conclusion (FIG. 9). In addition, due to poor biocompatibility, gold did not fit tightly with tissues, with a remarkable gap therebetween (d in FIG. 10), which is also the probabilistic off-target issue of gold markers in clinical use. However, PIDA was basically integrated into the tissues, so that a high degree of healing was achieved, and detachment did not tend to occur (f in FIG. 10).

| | Reference | Gold marker | PIDA | Control |
|---|---|---|---|---|
| | range | group | group | group |
| ALT (U/L) | 33.70-98.70 | 97.8 | 59 | 68.2 |
| AST (U/L) | 69.70-322.90 | 406 | 192.2 | 191.5 |
| ALP (U/L) | 1.30-211.00 | 345 | 138 | 162 |
| CREA (U/L) | 19.43-64.97 | 29 | 17 | 17 |

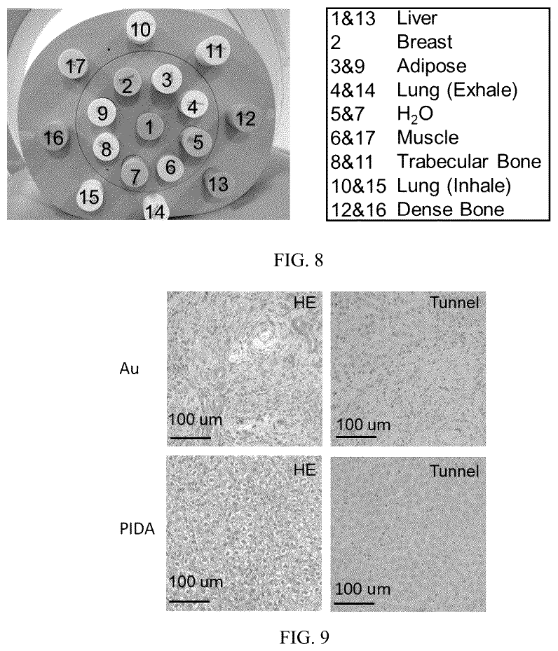

In order to get closer to an actual clinical application scenario, an original human body cyberknife treatment model accompanied with a clinical cyberknife instrument was used in the present invention (FIG. 8). Each movable cylinder in this model corresponded to the CT signal intensity corresponding to a real human organ, so that the CT imaging effect and subsequent radiation dose distribution planning in a human body environment could be better simulated. PIDA and gold markers were attached to the surfaces of the cylinders representing different organs of the human body (c in FIG. 7). The result showed that PIDA could favorably achieve local CT marking in the complex human body environment, and also exhibited high-quality artifact-free CT imaging (d in FIG. 7).

When confirming the radiation dose distribution of subsequent cyberknife treatment according to CT imaging distribution, the artifact-free CT marking of PIDA was closer to original actual requirements, while the artifact of the gold marker had a significant impact on peripheral dose distribution (e, f, and g in FIG. 7). Based on this, it is believed in the present invention that in addition to the high-efficiency CT imaging inherent in the gold markers, PIDA can further reduce side effects, improve the stability of the relative position and CT imaging quality, thereby providing better assurance for subsequent cyberknife treatment effects.

In order to further verify the performance of PIDA in actual cyberknife treatment, PIDA was implanted into a rat and a beagle respectively, and respiratory movement tracking and subsequent cyberknife treatment were performed on the rat and the beagle respectively according to clinical cyberknife patient treatment specifications. The entire process satisfied cyberknife tracking requirements, and final tracking of the rat.

One of the significant challenges for radiotherapy is to compensate for tumor movement caused by patient respiration. The International Commission on Radiation Units and Measurements (ICRU) recommends adding markers at a tumor location to compensate for the geometric uncertainty caused by this movement and tumor rotation. Based on current needs, stereotactic body radiotherapy (SBRT) based on advances in image-guided radiotherapy (IGRT) and movement management technology has been widely used. A cyberknife stereotaxic radiotherapy device (CNNC ACCURAY) introduces a fiducial tracking system that requires the use of fiducial markers (radio-opaque markers implanted around or inside tumors) and synchronized respiration tracking. The cyberknife could be adjusted in time as the location of a moving target changes. (a in FIG. 10)

The fiducial tracking system can quickly, accurately, objectively measure the location of a trackable fiducial, thereby facilitating accurate localization and targeting for a patient. Thus, the accuracy of fiducial-based IGRT can be improved, while maintaining fast, direct, and objective alignment. To verify the performance of PIDA markers in clinical cyberknife radiotherapy tracking, in the invention, cyberknife tracking radiotherapy was performed by using different model animals according to a cyberknife manual.

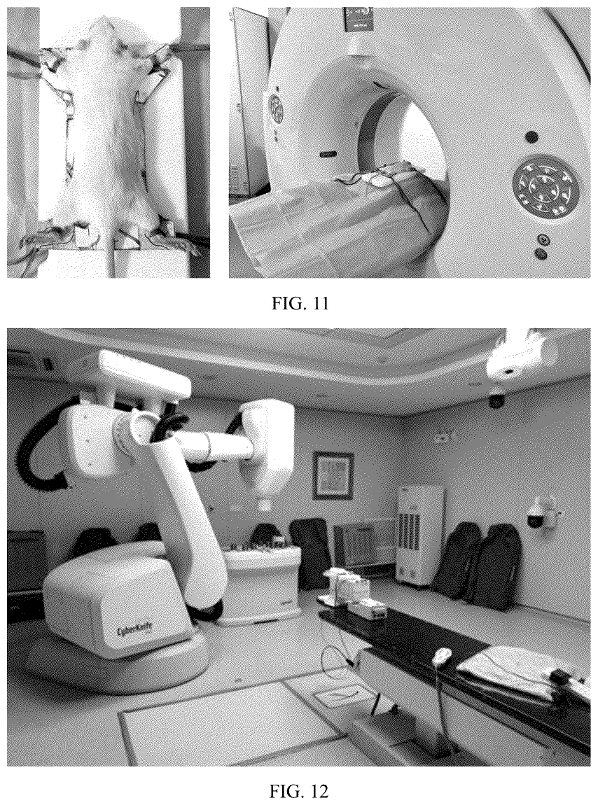

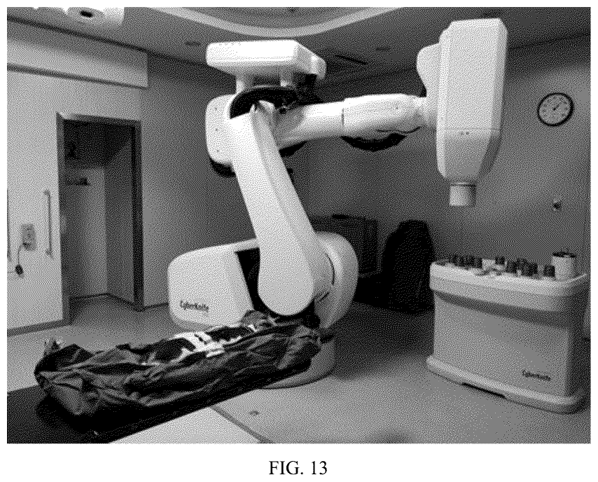

According to the shape of a standard Au marker, the PIDA marker was prepared as a cylinder with a diameter of 1 mm and a length of 3 mm (b in FIG. 10). Under the guidance of a CT image, the PIDA marker and the Au marker were implanted into leg muscles of a rat (FIG. 11). A human body respiratory movement simulation device was used to help the rat to simulate human body respiratory movement (FIG. 12). With respect to a correlated error, the difference between a target location estimated by a relevant model and an actual location determined by periodic X-ray imaging was quantified. The correlated error of the Au marker was 0.82±0.36 mm, and the correlated error of the PIDA marker was 0.57±0.19 mm. According to the cyberknife operating manual, the correlated error value should be kept at 5 millimeters or less, otherwise soft stop of the cyberknife would be triggered. As a replacement of the Au marker, the PIDA marker performed well in clinical cyberknife tracking in the simulated rat movement experiment.

The PIDA marker was implanted into the liver of a beagle according to the normal operation standard for cyberknife-implanted gold marker patients, and the surgery was performed by a professional surgeon. In order to ensure the consistency between preoperative CT modeling and the posture of the beagle during the cyberknife treatment, a memory air cushion designed for the posture of the cyberknife treatment patient was adopted here. The memory air cushion was soft in the initial state, and could be molded into a particular shape depending on the shape of the body lying inside. The memory air cushion became a substrate having a particular shape after gas is discharged (FIG. 13). The PIDA marker was clearly visible in a CT image of the liver of the beagle (g in FIG. 10), which was consistent with the result of the previous rat model. According to the CT result, a three-dimensional X-ray distribution map for beagle cyberknife radiotherapy was determined and designed (h in FIG. 10).

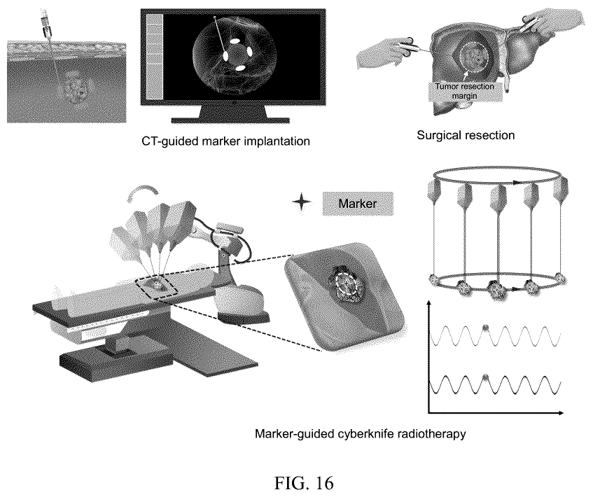

The PIDA marker in the liver of the beagle was tracked in real time during respiratory movement and matched with a constructed 3D model (i in FIG. 10). Due to real-time PIDA marker tracking, beams were modulated during free respiration. A regular respiratory movement curve of the beagle was tracked in real time (j in FIG. 10). The correlated error value (in millimeters) indicating the degree of consistency of a particular model point with a current synchronization model was 1.07±0.55 millimeters. It was reported that the correlated error value of the Au marker in a clinical cyberknife patient was 1.7±1.1 mm (k in FIG. 10). An uncertainty (%) parameter provided a detection uncertainty value of a fiducial extraction algorithm, and served as a metric fiducial configuration for extraction incorrectness. The uncertainty of the PIDA marker was 9.10±2.30% (l in FIG. 10), and a default value of an uncertainty (%) threshold parameter was 40% according to the cyberknife manual. The experimental result showed that the PIDA marker met clinical requirements in tracking the respiratory movement of the liver of the beagle during cyberknife radiotherapy. PIDA could achieve surgical treatment and cyberknife radiotherapy under the dual guidance of an image marker and naked eye observation (FIG. 16).

### Example 4: Biocompatibility and Biodegradability of PIDA Marker

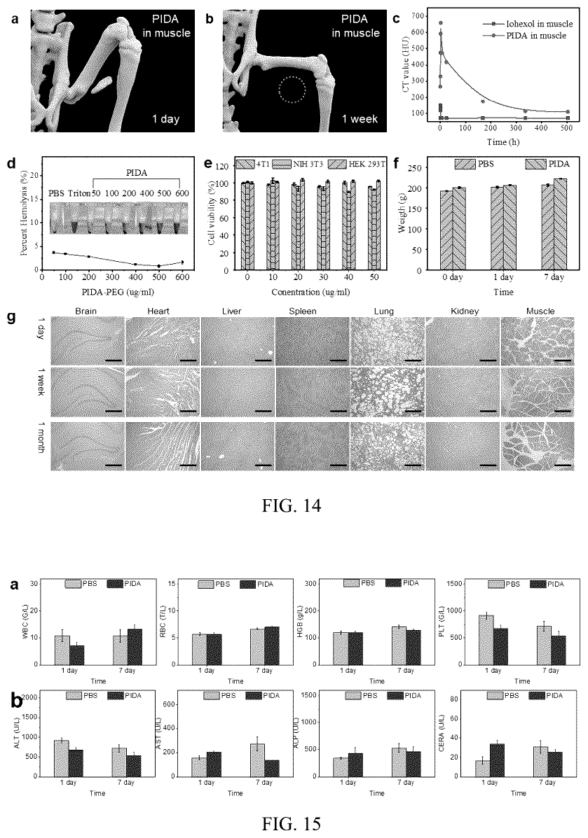

When a material is applied in the biomedical field, in addition to the special imaging/treatment effect of the material, the biocompatibility of the material itself is the topic that people are most concerned about. At the cell level, PIDA dispersions of different concentrations were mixed with rat erythrocytes in the present invention. A negative control PBS group showed no hemolysis, while a positive control Triton group showed complete hemolysis, so that the latter was set as a 100% hemolysis control. The hemolysis rates were all lower than 5% in PIDA groups of different concentrations, indicating that the corresponding PIDA did not cause breakage of red blood cells and did not cause hemolysis (d in FIG. 14). In addition, in the present invention, PIDA dispersions of different concentrations were respectively incubated together with 4T1 cells (mouse breast cancer cells), NIH 3T3 cells (mouse embryonic fibroblasts) and HEK 293T (human embryonic kidney cells) for 12 hours, and cell viability was verified by means of MTT verification. The result showed that none of the corresponding PIDA showed remarkable killing effect on the cells (e in FIG. 14).

At the animal level, after a 5 mg/ml PIDA dispersion was injected into the leg muscles of the rat, the weight of the rat increased normally, and the observed physiological state had no significant difference from that of the control group. At the same time, it was observed that CT signals of PIDA in muscles and tumors disappeared after seven days, indicating that a good CT imaging effect could be achieved during treatment, and PIDA degraded and disappeared after the treatment without causing any trouble or hidden danger to subsequent imaging treatment or even daily life (a, b, and c in FIG. 14). During the experiment, it was observed that the rat was in good condition and the weight increased normally, with no significant difference from the PBS control group (f in FIG. 14). The tissue sectioning result of major organs showed that PIDA did not exhibit corresponding systemic toxicity throughout the entire process (g in FIG. 14). As indicated by detection comparison between related indicators of blood routine (white blood cells, red blood cells, hemoglobin, and platelets) (a in FIG. 15) and related indicators of liver and kidney function tests (alanine aminotransferase, aspartate aminotransferase, alkaline phosphatase, and creatinine) (b in FIG. 15), there were no significant differences between relevant indicators of normal rats in the PIDA group and in the PBS control group (FIG. 15), indicating that there was no effect on the physiological state. Tissues at the injection site were taken out for Raman testing at different time points, and the result showed that a Raman signal of PIDA gradually decreased over time and finally disappeared completely. On the other hand, compared with liver implantation of PIDA solid fibers and gold markers, a liver tissue gold marker group at a corresponding implantation site showed remarkable cell necrosis, and corresponding liver and kidney function analysis in blood also showed obvious inflammatory response. However, no abnormality was observed in the PIDA group. In summary, PIDA had good biosafety and biodegradability.

PIDA was fixed to the body surface of the rat with a medical tape. The CT result showed that PIDA was clearly visible inside the body, and the location thereof was relatively fixed relative to various organs inside the body. PIDA moved on the body surface along with skin movement, and could reflect the respiratory movement of the rat itself to a certain extent, thereby providing tracking and identification functions for corresponding cyberknife treatment.

It should be easily understood by those skilled in the art that the foregoing description is only preferred embodiments of the present invention and is not intended to limit the present invention. All the modifications, identical replacements and improvements within the spirit and principle of the present invention should be in the scope of protection of the present invention.

1-10. (canceled)

11. A carbon-iodine conjugated polymer, wherein the structural formula of the carbon-iodine conjugated polymer is as shown in formula I:

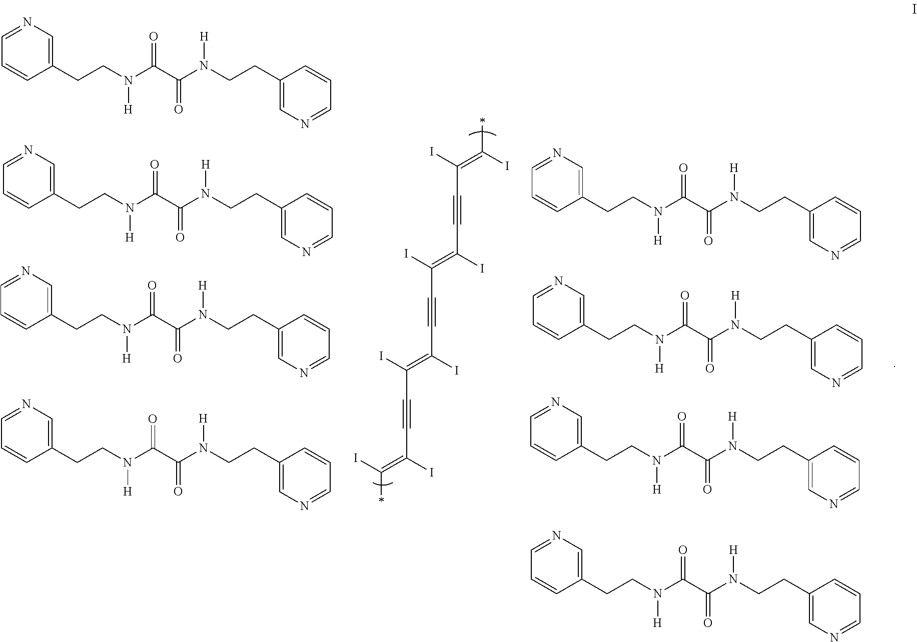

12. A synthesis method of a carbon-iodine conjugated polymer having the structure shown in formula I, wherein a reaction formula is as follows: 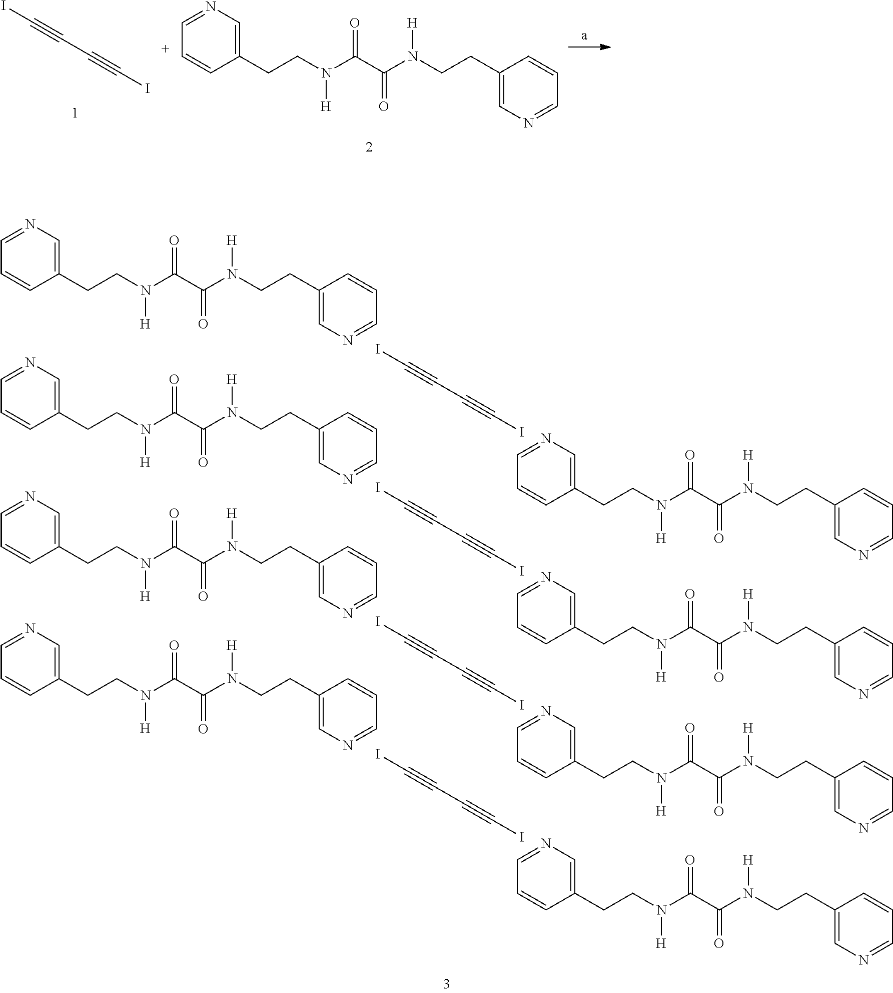 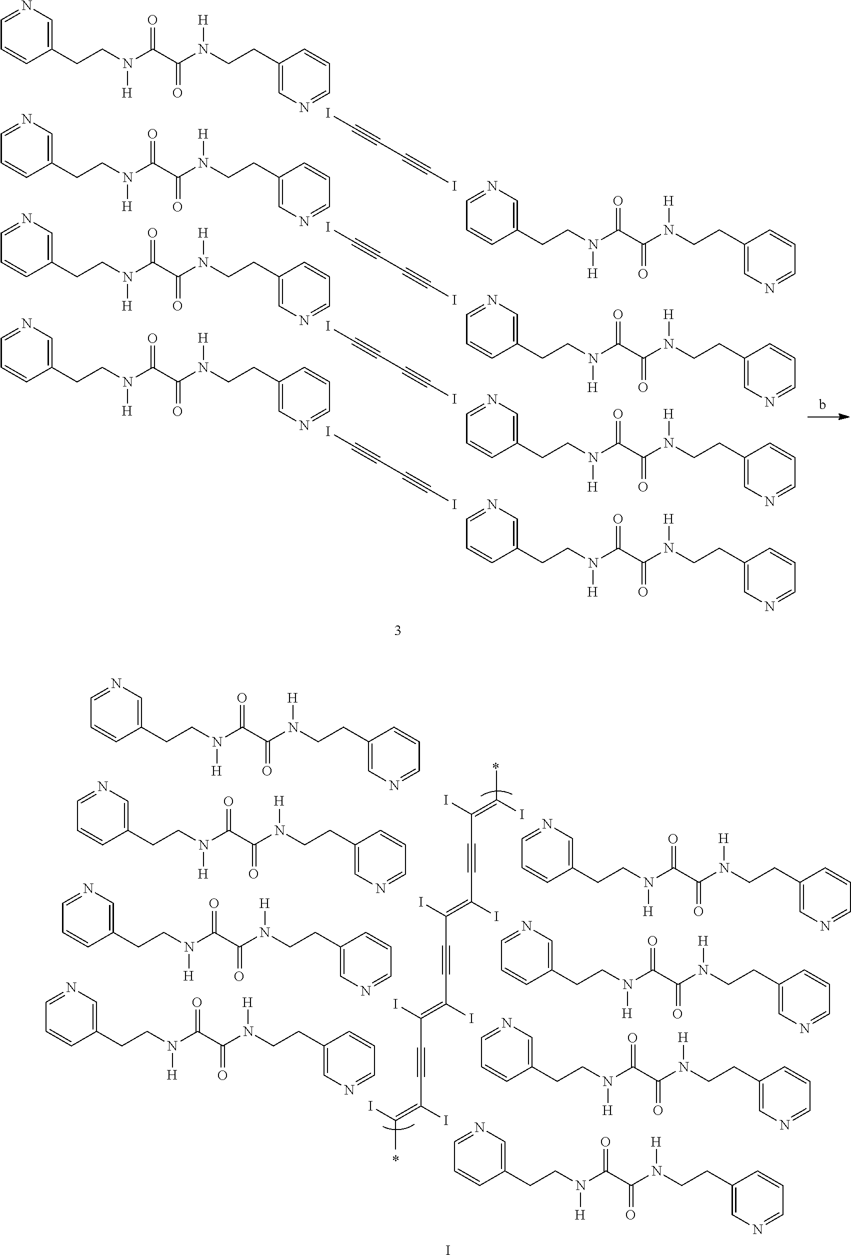
the synthesis method comprises the following steps:
a, after being added to methanol, ethanol, or isopropanol, the iodine substituted hexadine shown in formula 1 and the ligand shown in formula 2 are kept at −30° C. to −10° C. for 5 to 10 days, and then kept at 10° C. to 30° C. for 5 to 12 hours, the ligand and the iodine substituted hexadine being arranged regularly to form an intermediate shown in formula 3; and
b, carrying out topochemical polymerization on iodine atoms on the iodine substituted hexadine in formula 3 and pyridine group nitrogen atoms at a terminal of the ligand in formula 2 through halogen bonding to obtain a carbon-iodine conjugated polymer having the structure shown in formula I.

13. A synthesis method of a carbon-iodine conjugated polymer having the structure shown in formula II, wherein a reaction formula is as follows: 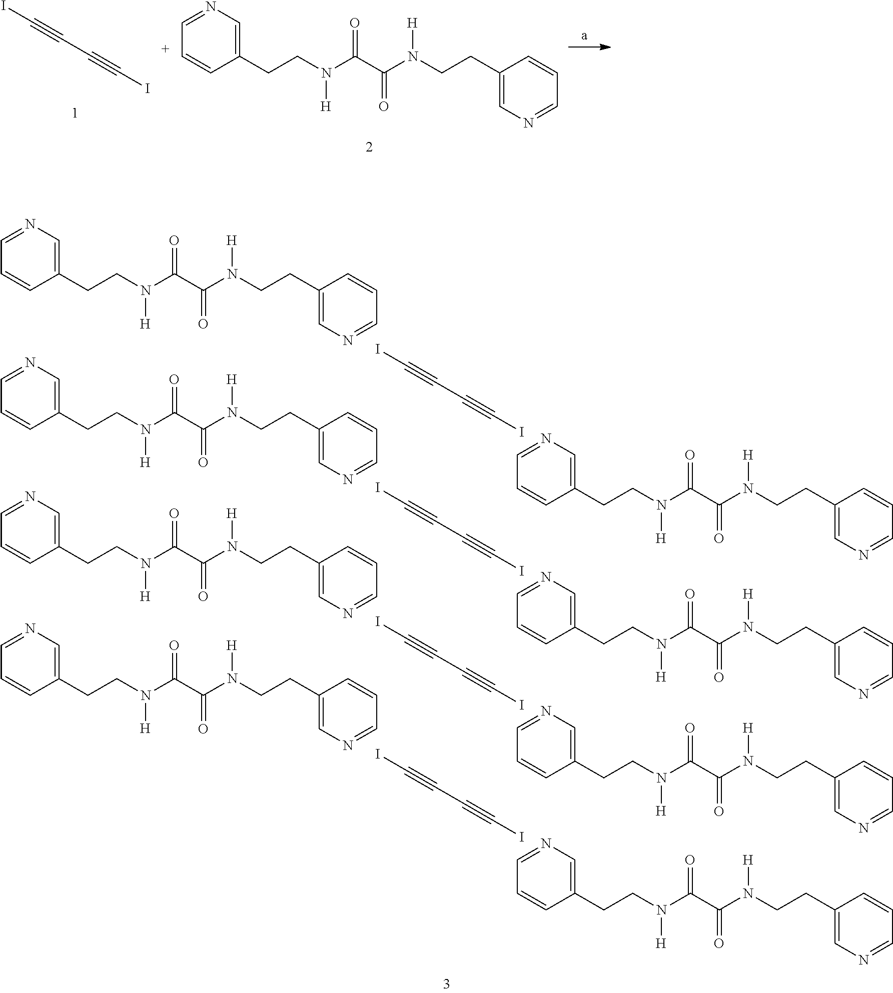 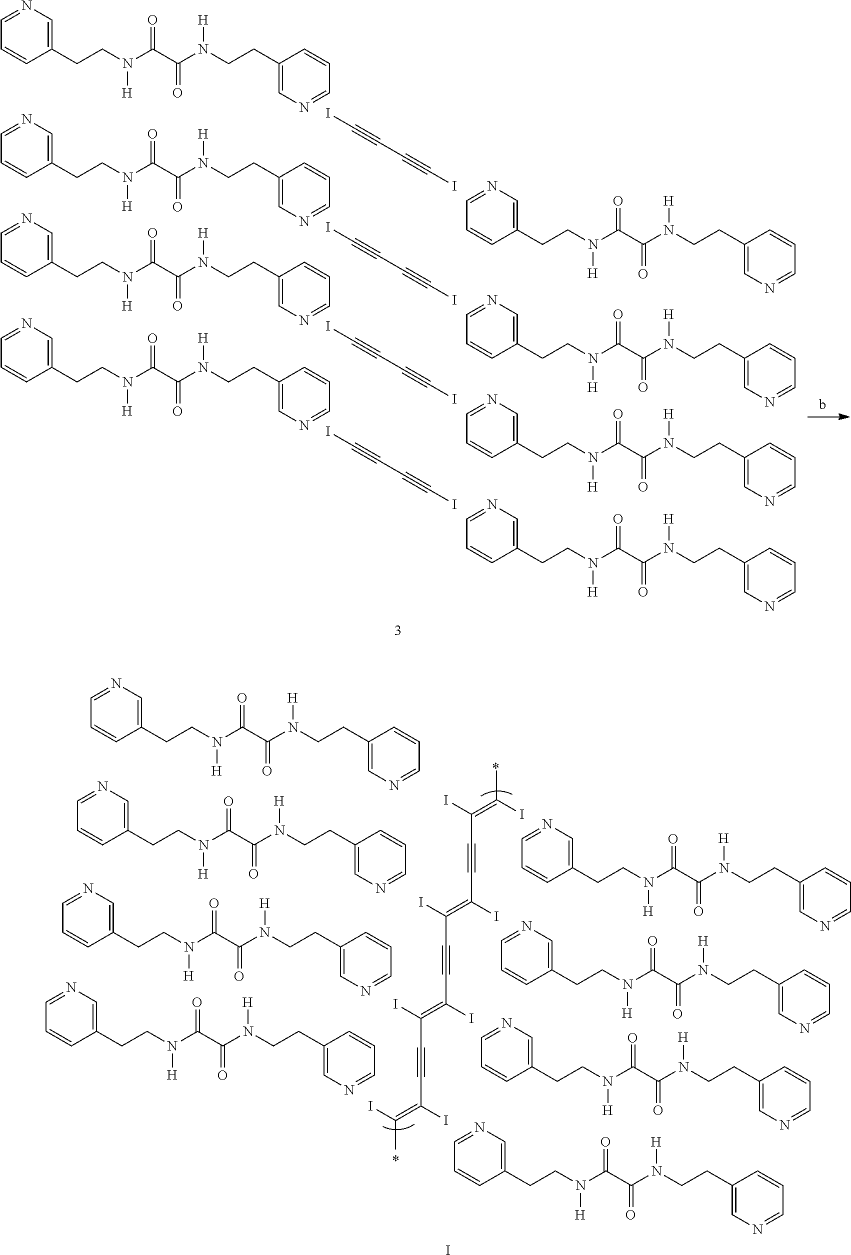 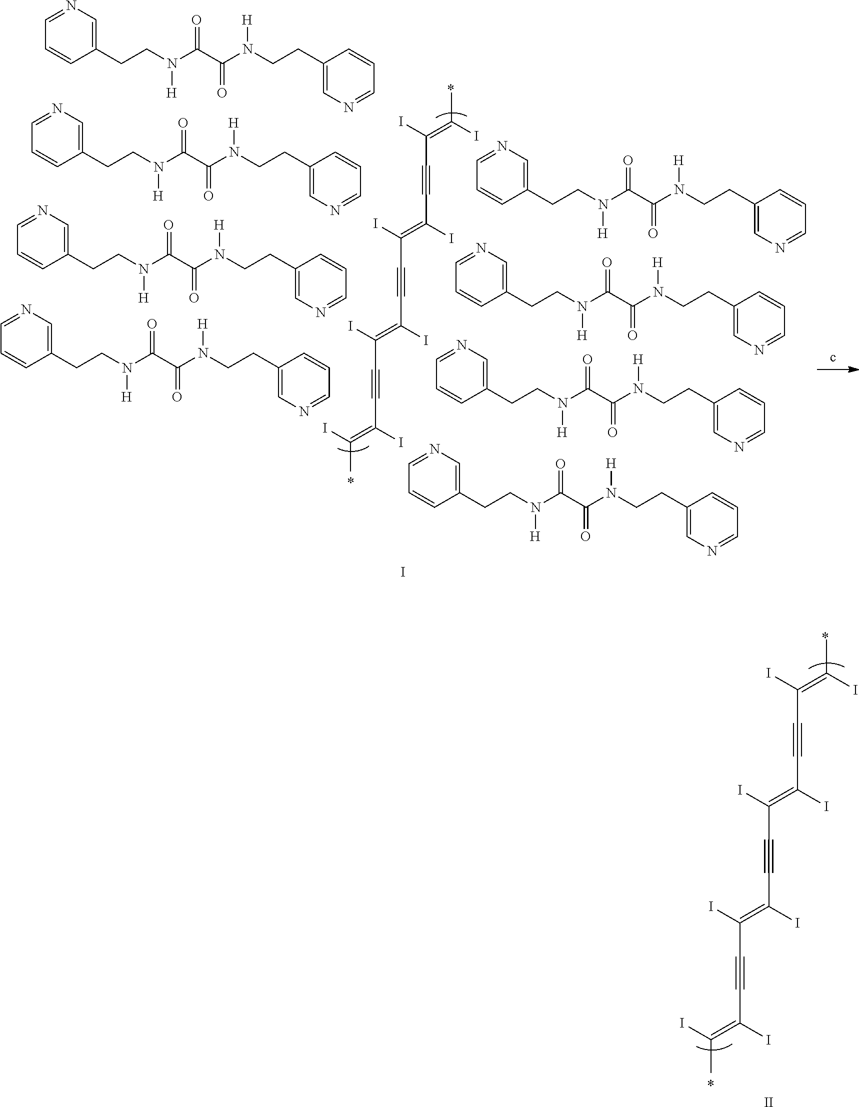
the synthesis method comprises the following steps:
a, after being added to methanol, ethanol, or isopropanol, the iodine substituted hexadine shown in formula 1 and the ligand shown in formula 2 are kept at −30° C. to −10° C. for 5 to 10 days, and then kept at 10° C. to 30° C. for 5 to 12 hours, the ligand and the iodine substituted hexadine being arranged regularly to form an intermediate shown in formula 3;
b, carrying out topochemical polymerization on iodine atoms on the iodine substituted hexadine in formula 3 and pyridine group nitrogen atoms at a terminal of the ligand in formula 2 through halogen bonding to obtain a carbon-iodine conjugated polymer having the structure shown in formula I; and
c, adding the carbon-iodine conjugated polymer having the structure shown in formula I and obtained in step b into methanol or a dilute hydrochloric acid solution and washing the same, performing a centrifugal operation to remove a supernatant, and carrying out vacuum drying to obtain a carbon-iodine conjugated polymer having the structure shown in formula II.

14. A method for preparing an aqueous dispersion containing a carbon-iodine conjugated polymer having the structure shown in formula I, wherein by: causing a carbon-iodine conjugated polymer having the structure shown in formula I and an amphiphilic polymer to undergo ultrasonic stripping in water, a hydrophobic end of the amphiphilic polymer being an alkyl chain having a carbon atomic number greater than or equal to 10, and a hydrophilic end thereof being polyethylene glycol; and causing the carbon-iodine conjugated polymer having the structure shown in formula II to be linked to the amphiphilic polymer through an intermolecular force to form an aqueous dispersion containing the carbon-iodine conjugated polymer having the structure shown in formula II, the carbon-iodine conjugated polymer having the structure shown in formula I and II being as follows:

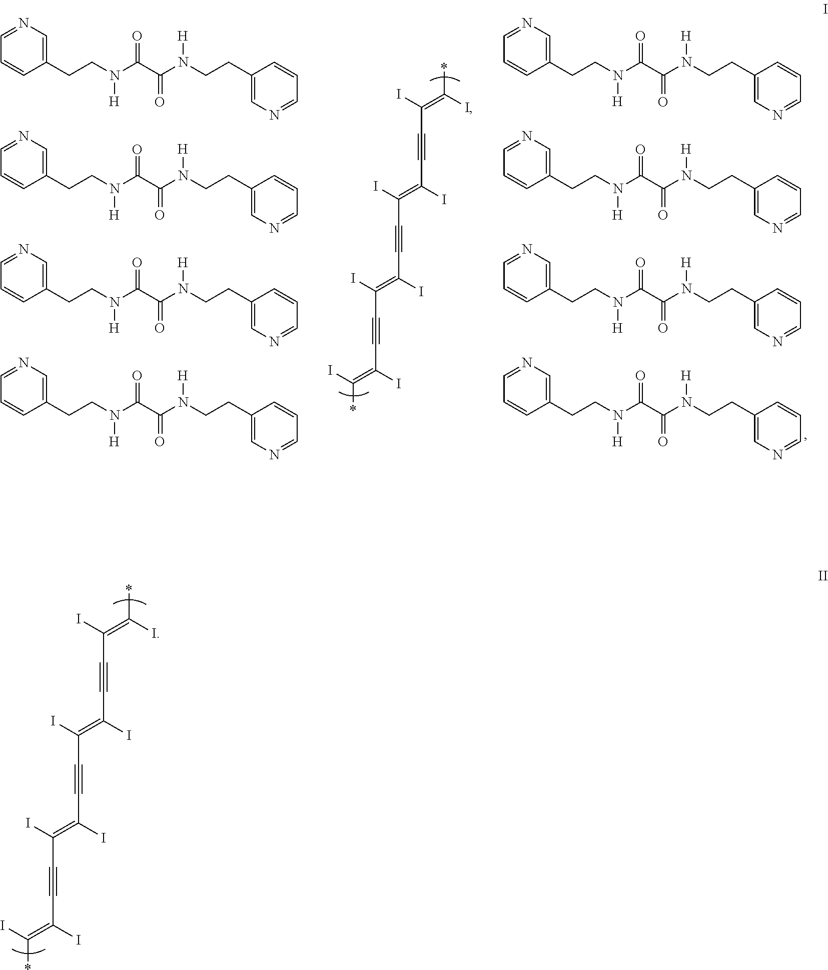

15. An aqueous dispersion containing a carbon-iodine conjugated polymer having the structure shown in formula II prepared by the method according to claim 14.

16. A use of the carbon-iodine conjugated polymer according to claim 11, wherein the use is specifically for preparing a localization marker.

17. The use according to claim 16, wherein the localization marker is an imaging marker.

18. The use according to claim 17, wherein the imaging marker is an X-ray marker.

19. The use according to claim 18, wherein the X-ray marker is a CT imaging marker.

20. A use of the carbon-iodine conjugated polymer according to claim 1, wherein the use is specifically for preparing a body surface auxiliary marker patch.
